SEQUENCE LISTING (16 sequences)

SEQ ID NO: 1 - protein, 26 aa, Homo sapiens
  Morgana epitope
LSELKPKFQEHIIQAPKPVEAIKRPS

SEQ ID NO: 2 - protein, 331 aa, Homo sapiens
  Morgana
MALLCYNRGCGQRFDPEANSDDACTYHPGVPVFHDALKGWSCCKRRTTDFSDFLSIVGCTKGRHNSEKPPEPVKPEVKTTEKKELSELKPKFQEHIIQAPKPVEAIKRPSPDEPMTNLELKISASLKQALDKLKLSSGSEEDKKEEDSDEIKIGTSCKNGGCSKTYQGLQSLEEVCVYHSGVPIFHEGMKYWSCCRRKTSDFNTFLAQEGCTRGKHVWTKKDAGKKVVPCRHDWHQTGGEVTISVYAKNSLPELSQVEANSTLLNVHIVFEGEKEFHQNVKLWGVIDVKRSYVTMTATKIEITMRKAEPMQWASLELPTTKKQEKQKDIAD

SEQ ID NO: 3 - protein, 5 aa, Mus musculus
  CDRH1
SYWMH

SEQ ID NO: 4 - protein, 17 aa, Mus musculus
  CDRH2
QIDPSDNYTSYNQKFKG

SEQ ID NO: 5 - protein, 5 aa, Mus musculus
  CDRH3
PYGSY

SEQ ID NO: 6 - protein, 16 aa, Mus musculus
  CDRL1
RSSQSLVHSNGNSYLH

SEQ ID NO: 7 - protein, 7 aa, Mus musculus
  CDRL2
KVSNRFS

SEQ ID NO: 8 - protein, 9 aa, Mus musculus
  CDRL3
SQSTHVPLT

SEQ ID NO: 9 - protein, 114 aa, Mus musculus
  Heavy chain variable region
QVQLQQPGAELVKPGASVKLSCKASGYTFTSYWMHWVKQRPGQGLEWIGQIDPSDNYTSYNQKFKGKATLTVDTSSSTAYMQLSSLTSEDSAVYYCASPYGSYWGQGTLVTVSA

SEQ ID NO: 10 - protein, 438 aa, Mus musculus
  Heavy chain
QVQLQQPGAELVKPGASVKLSCKASGYTFTSYWMHWVKQRPGQGLEWIGQIDPSDNYTSYNQKFKGKATLTVDTSSSTAYMQLSSLTSEDSAVYYCASPYGSYWGQGTLVTVSASKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 11 - protein, 114 aa, Mus musculus
  Light chain variable region
DVVMTQTPLSLPVSLGDQASISCRSSQSLVHSNGNSYLHWYLQNPGQSPKLLIYKVSNRFSGVPDRFSGSGSGTDFTLKISRVEAEDLGVYFCSQSTHVPLTFGAGTKLELKRA

SEQ ID NO: 12 - protein, 219 aa, Mus musculus
  Light chain
DVVMTQTPLSLPVSLGDQASISCRSSQSLVHSNGNSYLHWYLQNPGQSPKLLIYKVSNRFSGVPDRFSGSGSGTDFTLKISRVEAEDLGVYFCSQSTHVPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 13 - DNA, 1374 bp, Mus musculus
  Heavy chain (includes signal sequence and stop codon)
atgggatggagctgtatcatcctcttcttggtatcaacagctacaggtgtccactcccaggtccaactgcagcagcctggggctgagcttgtgaagcctggggcttcagtgaagctgtcctgcaaggcttctggctacaccttcacctcctactggatgcactgggtgaaacagaggcctggacaaggccttgagtggatcggacagattgatccttctgataattatactagctacaatcaaaaattcaagggcaaggccacattgactgtagacacatcctccagcacagcctacatgcaactcagcagcctgacatctgaggattctgcggtctattactgtgcaagtccgtatggtagttactggggccaagggactctggtcactgtctctgcatccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 14 - protein, 457 aa, Mus musculus
  Heavy chain (includes signal peptide)
MGWSCIILFLVSTATGVHSQVQLQQPGAELVKPGASVKLSCKASGYTFTSYWMHWVKQRPGQGLEWIGQIDPSDNYTSYNQKFKGKATLTVDTSSSTAYMQLSSLTSEDSAVYYCASPYGSYWGQGTLVTVSASKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - DNA, 717 bp, Mus musculus
  Light chain (includes signal sequence and stop codon)
atgaagttgcctgttaggctgttggtgctgatgttctggattcctgcttccagcagtgatgttgtgatgacccaaactccactctccctgcctgtcagtcttggtgatcaagcctccatctcttgcagatctagtcagagccttgtacacagtaatggaaactcctatttacattggtacctgcagaacccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 16 - protein, 238 aa, Mus musculus
  Light chain (includes signal peptide)
MKLPVRLLVLMFWIPASSSDVVMTQTPLSLPVSLGDQASISCRSSQSLVHSNGNSYLHWYLQNPGQSPKLLIYKVSNRFSGVPDRFSGSGSGTDFTLKISRVEAEDLGVYFCSQSTHVPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2023/0365664 A1
(43) Pub. Date: Nov. 16, 2023

(54) ANTI-MORGANA MONOCLONAL ANTIBODY FOR THE TREATMENT OF TUMORS

(71) Applicant: UNIVERSITA' DEGLI STUDI DI TORINO, Torino (IT)

(72) Inventors: Mara BRANCACCIO, Saint-Vincent (Aosta) (IT); Federica FUSELLA, Grugliasco (Torino) (IT); Laura SECLI', Parabita (Lecce) (IT); Emilia TURCO, Torino (IT)

(21) Appl. No.: 18/026,147

(22) PCT Filed: Sep. 14, 2021
(86) PCT No.: PCT/EP2021/075175

§ 371 Date: Mar. 14, 2023

(30) Foreign Application Priority Data
Sep. 14, 2020 (IT) 102020000021667

(51) Int. Cl. C07K 16/18; C12N 15/63; A61P 35/04

(52) U.S. Cl. CPC C07K 16/18; C12N 15/63; A61P 35/04; C07K 2317/24; C07K 2317/565

(57) ABSTRACT

A monoclonal antibody or an antibody fragment thereof capable of recognizing and binding to a specific epitope of the extracellular Morgana protein is provided. The monoclonal antibody or antibody fragment thereof inhibits growth of tumors secreting the extracellular Morgana protein and formation of metastases. An isolated nucleic acid comprising the nucleotide sequence encoding the monoclonal antibody or antibody fragment thereof, an expression vector including the isolated nucleic acid and the related host cell, and a pharmaceutical composition comprising the monoclonal antibody or antibody fragment or isolated nucleic acid are also provided.

The present invention falls within the field of antitumor therapy.

In particular, the invention relates to a monoclonal antibody or an antibody fragment, capable of binding a protein specifically secreted by tumor cells and effective in inhibiting tumor growth and/or the formation of metastases.

The invention also relates to an isolated nucleic acid comprising a nucleotide sequence encoding the aforementioned antibody or antibody fragment, an expression vector comprising the aforementioned encoding nucleotide sequence, a host cell including the aforementioned expression vector and a pharmaceutical composition comprising the aforementioned antibody or antibody fragment or nucleotide sequence encoding therefor.

Immunological therapies are notoriously a valid therapeutic approach for the treatment of various types of tumors. However, these therapies are only effective in a percentage of cancer patients. In addition, it often happens that immunotherapeutic agents effective in inhibiting the growth of a tumor are not able to inhibit the formation of metastases.

Consequently, researchers are constantly looking for alternative therapeutic approaches to increase the success rates of anticancer immunotherapeutic treatments. There is therefore a constant need to find new immunotherapeutic agents, which are effective even in patients that do not respond to the already available immunotherapies and which are able not only of inhibiting tumor growth but also of counteracting the migration of tumor cells and therefore the formation of metastases.

These and other needs are satisfied by the present invention, which provides a monoclonal antibody or antibody fragment thereof, which recognizes and binds to a particular epitope of the extracellular Morgana protein.

The studies carried out by the present inventors, which will be illustrated in detail below, have shown that the Morgana protein, a cytosolic protein expressed in a ubiquitous manner already described in the literature and which notoriously plays an important role as a regulator of various intracellular signaling cascades (1-3), also exists in an extracellular form. Studies carried out by the inventors have shown that this extracellular form of the Morgana protein is secreted by different types of human and mouse cancer cells—including breast, lung, colon and melanoma cancer cell lines—but not by non-cancerous cell lines. In addition, the inventors observed that the extracellular Morgana protein is capable of inducing the migration of tumor cells and that the treatment of subjects affected by tumors secreting the extracellular Morgana protein with the aforementioned monoclonal antibody, capable of binding an epitope of the extracellular form of Morgana, inhibits both tumor growth and metastasis formation. These properties make the aforementioned monoclonal antibody an extremely promising therapeutic tool for anticancer treatment.

The present invention therefore relates to a monoclonal antibody or antibody fragment thereof, which binds to the extracellular Morgana protein at the epitope having the amino acid sequence SEQ ID NO: 1. The expression “antibody fragment thereof” refers to immunoglobulin fragments that maintain the binding ability of the monoclonal antibody from which they are derived.

The monoclonal antibody or antibody fragment thereof according to the invention can be monospecific or bispecific. It can also be humanized. It is known that antibody humanization is used to reduce the immunogenicity of an animal monoclonal antibody and to improve its activity in the human immune system. There are different strategies for the humanization of monoclonal antibodies, which are per se known to the person skilled in the art. By way of example, antibodies humanized through the CDR grafting technique, chimeric antibodies and fully humanized antibodies are mentioned. A review of some of the techniques available for the humanization of antibodies is available in (1).

In addition, the monoclonal antibody of the invention may possibly be conjugated to an appropriate drug or toxin, selected from the drugs and toxins known per se for antitumor use.

The antibody fragment that falls within the scope of the invention is a fragment that maintains the ability to bind to the extracellular Morgana protein epitope having the amino acid sequence SEQ ID NO: 1. Preferred antibody fragments are Fab, Fab′, F(ab′)2, scFv, dsFv, ds-scFv, minibodies, diabodies, and their dimers, multimers or bispecific antibody fragments.

The amino acid sequence of the Morgana protein is known per se and is illustrated in SEQ ID NO: 2.

The invention also relates to an isolated nucleic acid, preferably DNA, comprising a nucleotide sequence encoding the aforementioned monoclonal antibody or antibody fragment, as well as an expression vector comprising the nucleotide sequence encoding the monoclonal antibody or antibody fragment of the invention.

The scope of the invention also comprises a host cell including the expression vector comprising the nucleotide sequence encoding the monoclonal antibody or antibody fragment of the invention. The host cell of the invention is used for the production of the monoclonal antibody or antibody fragment by known recombinant technologies, the implementation of which falls within the skills of the person skilled in the art.

Also included in the scope of the invention is a hybridoma producing the monoclonal antibody or antibody fragment of the invention.

As previously indicated, the monoclonal antibody or antibody fragment of the invention has the ability to inhibit the growth of tumors secreting the extracellular Morgana protein, as well as the formation of metastases. Therefore, it is suitable for use as a medicament, in particular as an immunological therapy for the treatment of tumors secreting the extracellular Morgana protein, such as breast cancer, lung cancer, colon cancer and melanoma.

A particularly advantageous property of the monoclonal antibody or antibody fragment of the invention is its efficacy against triple negative breast cancer and non-small cell lung cancer cell lines, which are tumors that are very difficult to treat with currently available therapies; this makes it particularly valuable as a therapeutic tool.

Therefore, the scope of the invention comprises the use as a medicament of the monoclonal antibody or antibody fragment of the invention and of the nucleotide sequence encoding therefor. The preferred therapeutic applications concern the therapeutic treatment of the tumor pathologies specified above. In such therapeutic applications, the monoclonal antibody or antibody fragment of the invention or the nucleotide sequence encoding therefor can optionally be used in a combined therapy with other antitumor agents, for example in combination with immune checkpoint inhibitors, such as for example blocking antibodies against PD-1, PD-L1, CTLA-4 or LAG-3.

For use in the therapeutic field, the monoclonal antibody or antibody fragment or nucleotide sequence of the invention are formulated in a pharmaceutical composition which also includes suitable pharmaceutically acceptable excipients, carriers, buffers and/or stabilizers. The specific composition of the pharmaceutical composition of the invention depends on various factors, such as for example the pathology to be treated, the indicated route of administration, the dosage regimen and others, which are known to those skilled in the art. The determination of the required dose of monoclonal antibody or antibody fragment or nucleic acid is also within the capabilities of the person skilled in the art.

Further features of the invention are defined in the dependent claims, which form an integral part of the present description.

The invention is described in greater detail in the experimental section that follows, which is provided only for illustrative purposes and is not intended to limit the scope of the invention, as defined by the appended claims.

The experimental section refers to the appended drawings, in which:

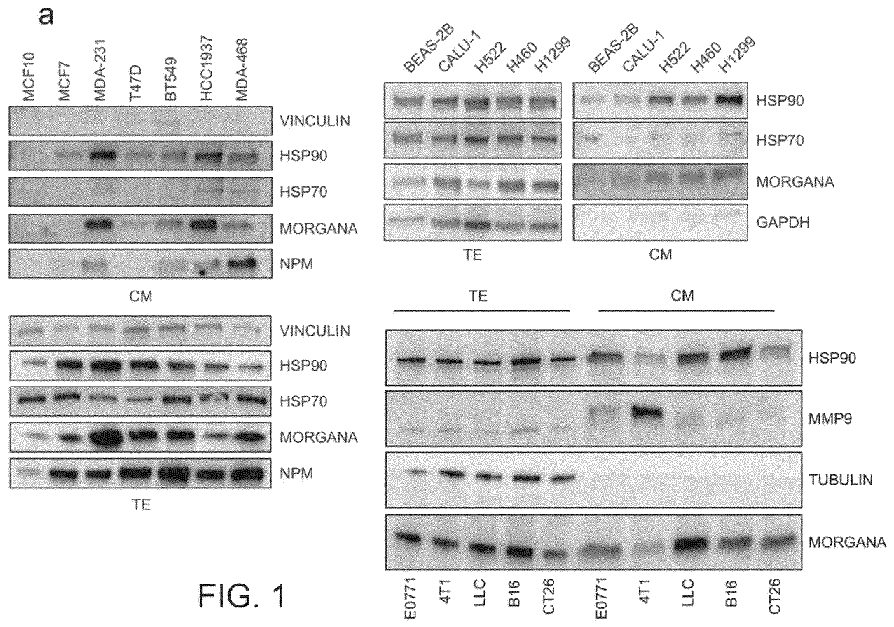

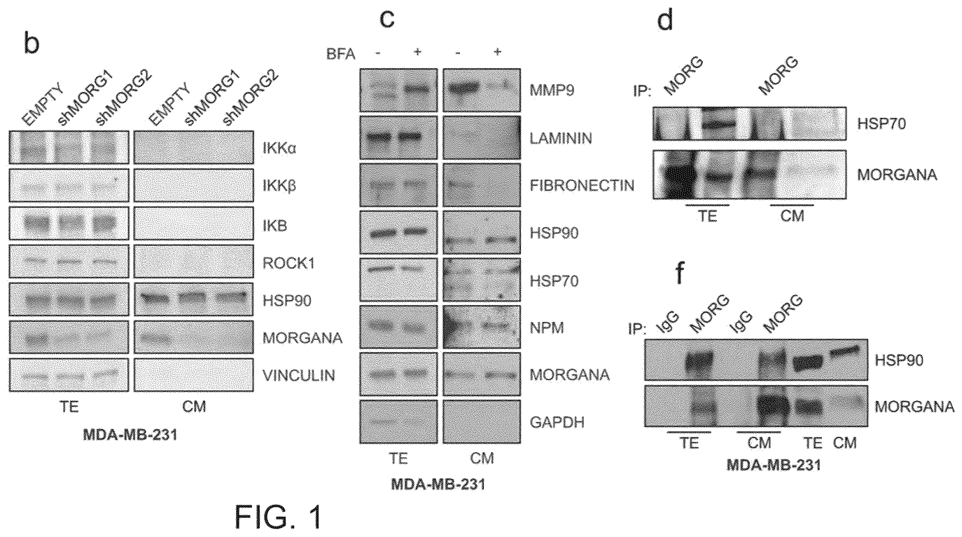

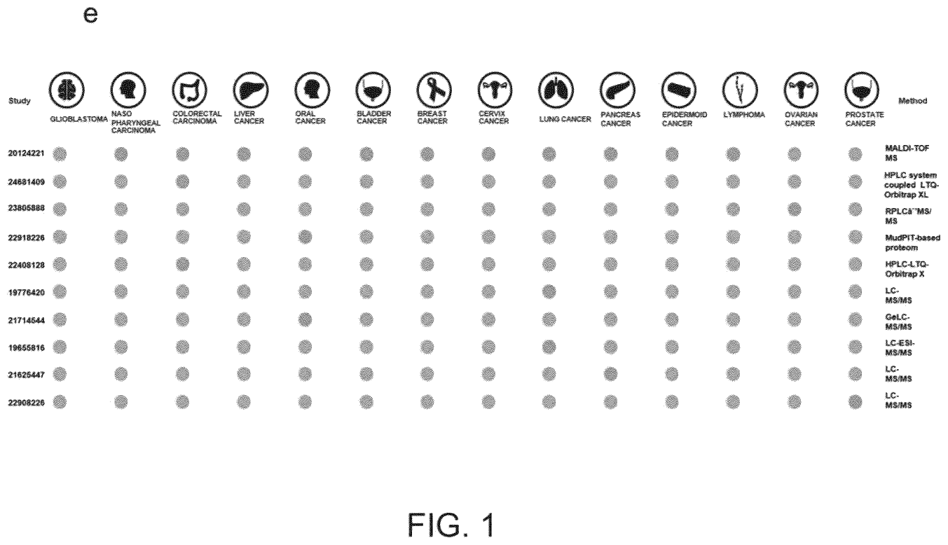

FIG. 1 presents the experimental results demonstrating that the Morgana protein is secreted specifically by tumor cells. Specifically: A) Western blot of cell extracts (TE) and conditioned medium (CM) of various human breast (left), lung (central) and murine tumor (right) and non-tumor cells showing that a relevant amount of Morgana is secreted only by cancer cells. B) Western blot of cell extracts (TE) and conditioned medium (CM) of MDA-MB-231 infected with an empty vector (EMPTY) and with two different shRNAs against Morgana (shMORG1 and shMORG2). The absence of known Morgana interactors in the conditioned medium demonstrates that the secretion is specific and not caused by the release of the cytoplasmic content secondary to cell damage. C) Western blot of cell extracts (TE) and conditioned medium (CM) of MDA-MB-231 treated or not with Brefeldin A (10 μg/ml for 5 hours). D) Immunoprecipitation of Morgana carried out from the conditioned medium without the use of detergent followed by Western blot analysis. E) Table obtained from the Human Cancer Secretome Database (6) in which it is evident that Morgana is secreted by lines of different types of tumor. F) Co-immunoprecipitation of Morgana and HSP90 from total extracts (TE) and conditioned medium (CM) obtained from MDA-MB-231.

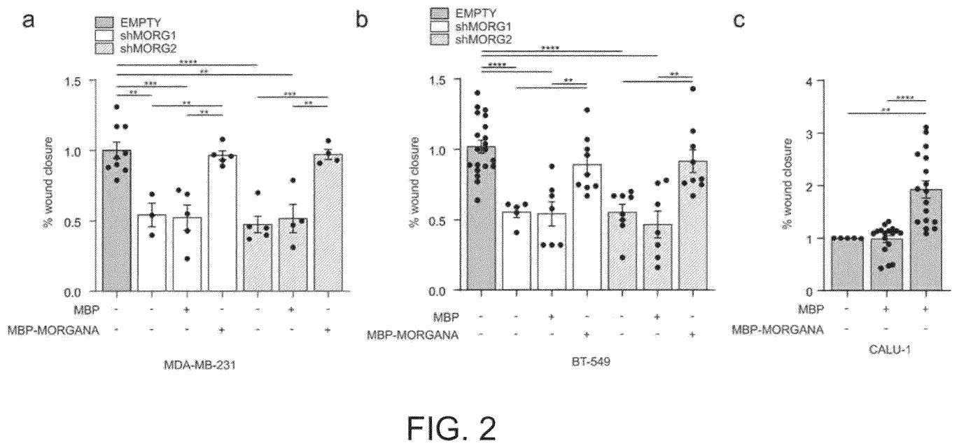

FIG. 2 presents the experimental results demonstrating that the extracellular Morgana protein induces migration of tumor cells. Specifically: A) Migration assay (wound healing) performed on control (EMPTY) or Morgana-interfered (shMORG1 and shMORG2) MDA-MB-231 tumor cells treated or not with the recombinant proteins MBP-Morgana or MBP as control. B) Migration assay (wound healing) performed on control (EMPTY) or Morgana-interfered (shMORG1 and shMORG2) BT-549 tumor cells treated or not with recombinant MBP and MBP-Morgana proteins. C) Migration assay (wound healing) performed on CALU-1 tumor cells (EMPTY) treated or not with the recombinant proteins MBP and MBP-Morgana. The graphs represent the quantifications of the pictures at time zero (t=0) and after 24 hours (t=24) made with the Axio Vision software. (* p<0.05; ** p<0.01; *** p<0.001).

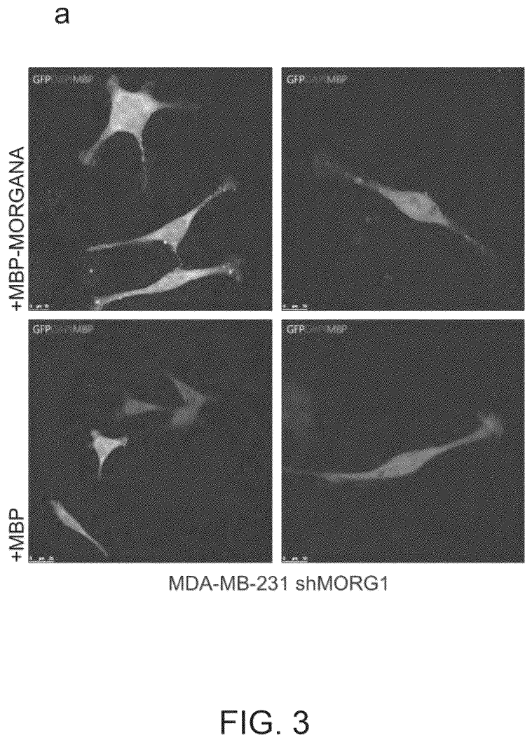

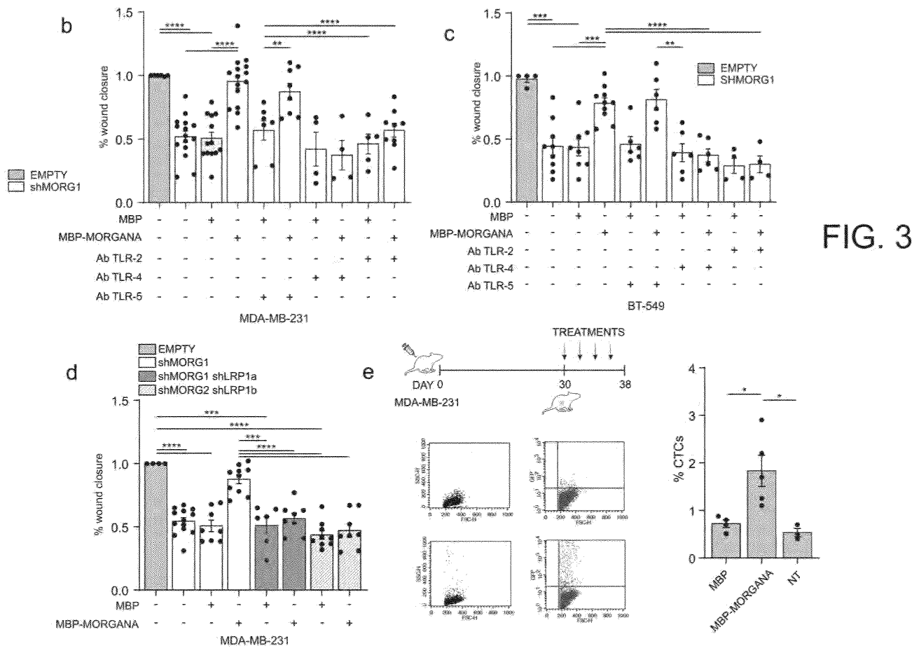

FIG. 3 presents the experimental results demonstrating that the extracellular Morgana protein induces the migration of tumor cells by binding the Toll-like receptors 2 and 4. Specifically: A) Immunofluorescence analysis performed using the anti-MBP antibody on MDA-MB-231 cells interfered for Morgana and treated with MBP or MBP-Morgana. B) Migration assay (wound healing) on MDA-MB-231 tumor cells treated or not with MBP or MBP-Morgana and in combination with blocking antibodies against TLR2, TLR4 or TLR5. The graph represents the quantification of cell migration carried out measuring wound at time zero (t=0) and after 24 hours (t=24) using AxioVision software. C) Migration assay (wound healing) on BT-549 tumor cells treated or not with MBP or MBP-Morgana and in combination with antibodies blocking TLR2, TLR4 or TLR5. The graph represents the quantification of cell migration carried out by analyzing the pictures of the wound at time zero and after 24 hours using AxioVision software. D) Migration assay (wound healing) on MDA-MB-231 tumor cells interfered for Morgana and LRP1, treated or not with MBP or MBP-Morgana. E) Timeline of the experiment. Immunodeficient NSG mice were inoculated subcutaneously with 1×10⁶ MDA-MB-231 cells infected with a vector containing GFP. After tumor growth, the animals were treated with either vehicle or 100 μg of recombinant MBP protein or 100 μg of recombinant MBP-MORGANA protein. The blood of the mice was analyzed by flow cytometry for the presence of positive GFP cells. (* p<0.05; ** p<0.01; *** p<0.001).

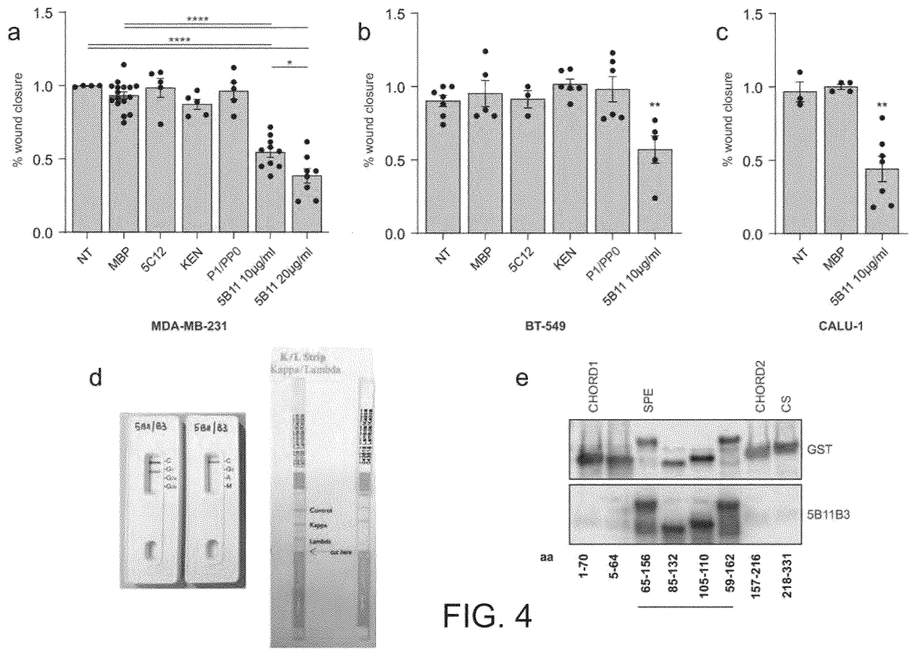

FIG. 4 presents experimental data on the characterization of the monoclonal antibody mAb 5B11B3. A) Migration assay (wound healing) performed on MDA-MB-231 tumor cells treated with different antibodies capable of recognizing Morgana. The anti-MBP antibody was used as a control. The 5B11B3 monoclonal antibody blocks cell migration in a dose-dependent manner. B) Migration assay (wound healing) performed on BT-549 tumor cells treated with different antibodies against Morgana. The anti-MBP antibody was used as control. The graph represents the quantification of wound closure carried out by measuring the wound at time zero and after 24 hours by AxioVision software. C) Migration assay (wound healing) on Calu-1 lung tumor cells treated with the 5B11B3 antibody or the control antibody. D) Characterization of the monoclonal antibody 5B11B3 using the Thermo Fisher kit (Pierce Rapid Isotyping kit mouse) and the Sigma kit (IsoQuick™ Kits for Mouse Monoclonal Isotyping). The antibody is IgG1 isotype and contains a kappa chain. E) Western blot performed on the different fragments of Morgana fused to GST using the mAb 5B11B3. All Morgana fragments containing amino acids 85-110 are recognized by the 5B11B3 antibody. (* p<0.05; ** p<0.01; *** p<0.001).

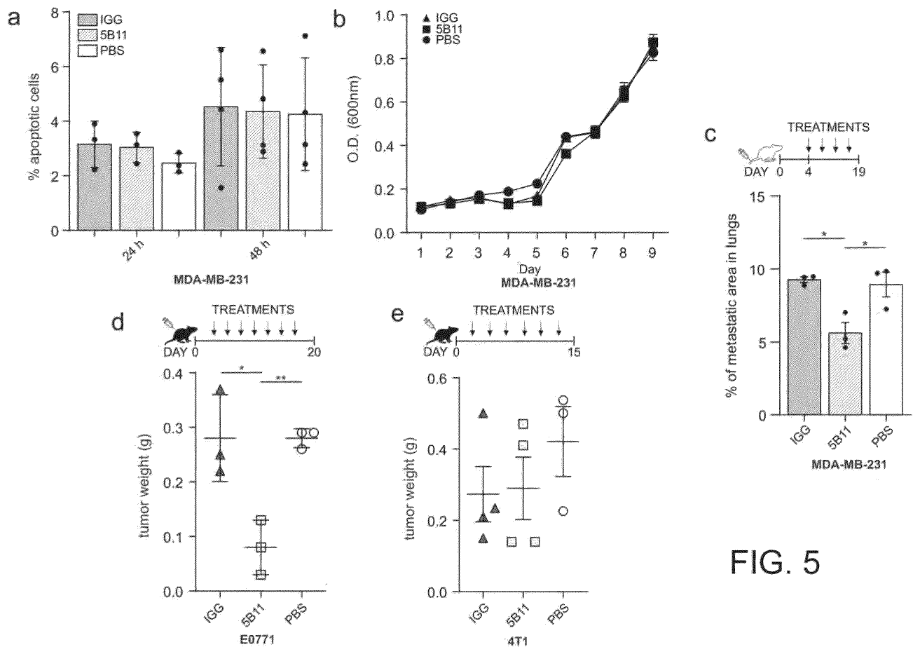

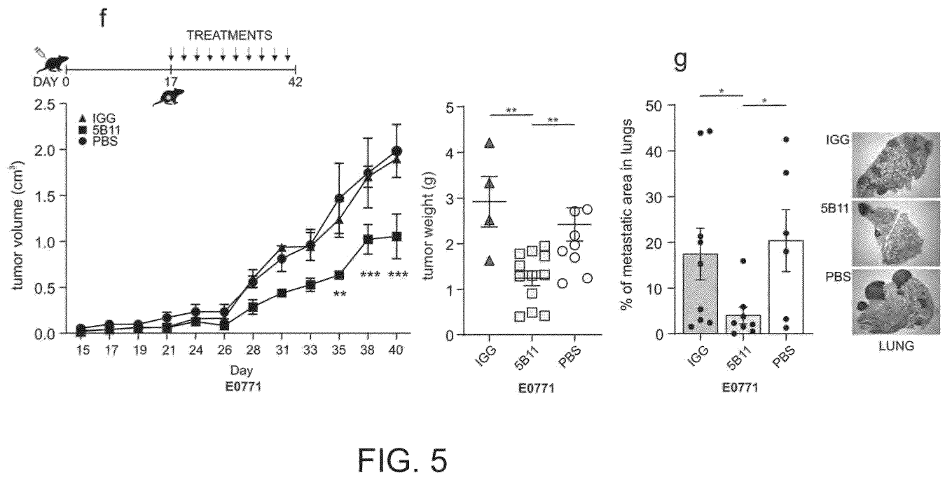

FIG. 5 presents the experimental results that demonstrate that the monoclonal antibody 5B11B3 of the invention is able to block tumor growth and the formation of metastases of breast cancer cells. Specifically: A) Analysis by flow cytometer after labeling with annexin V and propidium iodide of MDA-MB-231 cells cultured in vitro and treated with 5B11B3 or control antibodies or PBS for 24 and 48 hours. B) Proliferation assay on human breast cancer cells MDA-MB-231 treated with 5B 11B3 or control antibody or PBS C) Percentage of metastatic burden in the lungs of NSG immunocompromised mice inoculated with human breast cancer cells MDA-MB-231. The mice were treated with intravenous injections of 5B11B3 or control antibody or PBS. Timeline of the experiment is shown. D) C57BL/6 mice were subcutaneously inoculated with 200,000 E0771 breast cancer cells. From the day following the injection, the animals were treated with 100 μg of monoclonal antibody intravenously (5B11B3 or control antibody) 3 times a week for 20 days. Weight of tumors were obtained at the end of the experiment. Timeline of the experiment is shown. E) BALB/c mice were subcutaneously inoculated with 100,000 4T1 breast cancer cells. From the day following the injection, the animals were treated with 100 μg of monoclonal antibody intravenously (5B11B3 or control antibody) 3 times a week for 15 days. Weight of tumors were obtained at the end of the experiment. Timeline of the experiment is shown. F) C57BL/6 mice were subcutaneously inoculated with 200,000 E0771 breast cancer cells. The animals were treated with the 5B11B3 antibody or with the control antibody (100 μg intravenously, three times a week) starting from the moment when the tumor reached the size of 15 mm³ (15-17 days after the injection). Weight of tumors were obtained at the end of the experiment. Timeline of the experiment is shown. G) Images of lungs sections stained with hematoxylin and eosin used to evaluate and count metastases. The graph represents the average of the number of metastases present within the lung sections. (* p<0.05; ** p<0.01; *** p<0.001).

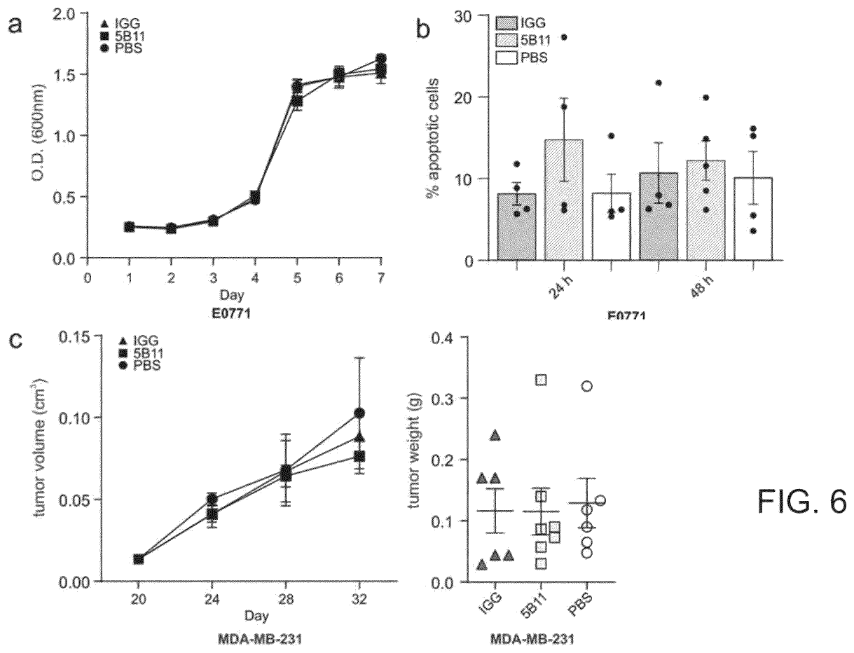

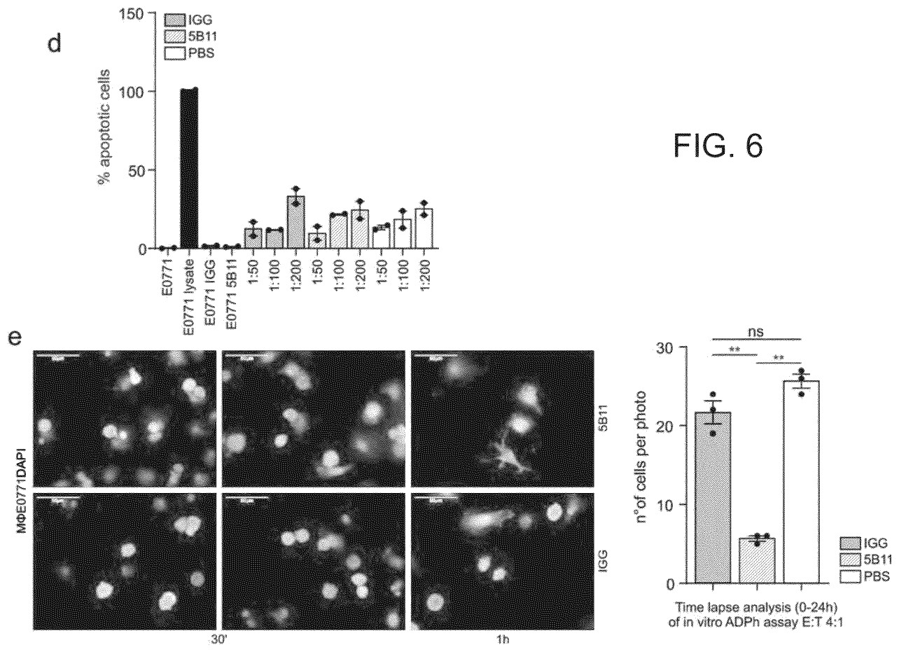

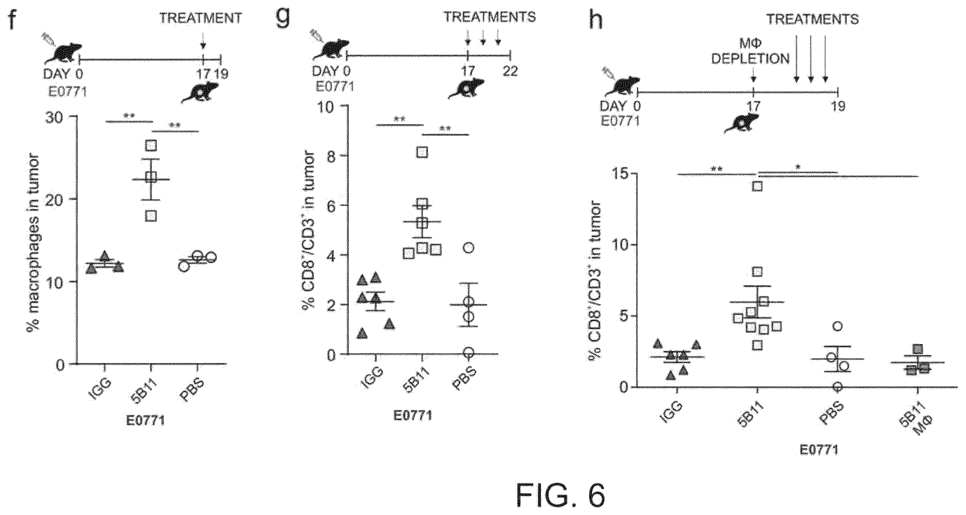

FIG. 6 presents the experimental results showing that treatment with the 5B11B3 antibody causes a greater recruitment of macrophages and CD8⁺ T lymphocytes in the primary tumor of a preclinical model of breast cancer. A) Proliferation assay on E0771 mouse breast cancer cells treated with mAb 5B11B3 or control antibody or PBS. B) Analysis by flow cytometer after labeling with annexin V and propidium iodide of E0771 cells cultured in vitro and treated with 5B11B3 or control antibodies or PBS for 24 and 48 hours. C) Volume and weight of tumors of immunodeficient NSG mice inoculated subcutaneously with 1×10⁶ MDA-MB-231 cells. Starting on day 20 (when the tumor was palpable) mice were treated with mAb 5B11B3 or control IgG every other day for 12 days. D) ADCC experiment. In vitro co-culture between splenocytes derived from the spleen of a C57BL/6 mouse (E: effectors) and E0771 tumor cells labeled with CFSE (T: target) at different ratios in the presence of the 5B11B3 antibody or the control antibody. The co-culture was maintained for 20 hours at 37° C. At the end of the experiment, the viability of the tumor cells was assessed by flow cytometry. E) In vitro co-culture experiment of E0771 cells with macrophages derived from the bone marrow of C57BL/6 mice. The graph represents the number of tumor cells after 24 hours of co-culture in the presence of the 5B11B3 antibody or the control antibody. (E=effector, macrophages; T=target, E0771 tumor cells). F) Flow cytometer analysis of macrophages recruited into the E0771-derived tumors after only 1 treatment of mAb 5B11B3 or control antibody. Timeline of the experiment is shown G) Flow cytometer analysis of CD8⁺ T lymphocytes recruited into E0771-derived tumors after 3 treatments with 5B11B3 or control antibody. Timeline of the experiment is shown. H) Percentage of CD8⁺ T lymphocytes present within the tumors of C57BL/6 mice inoculated subcutaneously with 200,000 E0771 tumor cells. When tumors reached 15 mm³ in size (15-17 days after injection), mice were treated intraperitoneum with clodronate liposomes. After 3 days they were treated 3 times with mAb 5B11B3 or control antibody. Timeline of the experiment is shown. (* p<0.05; ** p<0.01; *** p<0.001).

FIG. 7 presents the experimental results showing that the monoclonal antibody 5B11B3 is able to block the growth of tumors derived from murine colon cancer cells. A) The graph represents tumor measurements of BALB/c mice inoculated subcutaneously with 200,000 CT26 mouse colon cancer cells. When the tumors reached a size of 15 mm³, the animals were treated with 100 μg of monoclonal antibody intravenously (5B11B3 or control antibody) 3 times per week for 20 days. Timeline of the experiment is shown. B) Percentages of macrophages present in CT26-derived tumors after 1 treatment with 5B11B3 or control antibody obtained by flow cytometry. Timeline of the experiment is shown C) Analysis by flow cytometer of CD8⁺ T lymphocytes recruited into CT26-derived tumors after 3 treatments of mAb 5B11B3 or control antibody. Timeline of the experiment is shown. D) Percentage of CD8⁺ T lymphocytes present within the tumors of BALB/c mice inoculated subcutaneously with 200,000 CT26 colon cancer cells. When tumors reached 15 mm³ in size, mice were treated intraperitoneum with clodronate liposomes. After 3 days they were treated 3 times with mAb 5B11B3 or control antibody. Timeline of the experiment. (* p<0.05; ** p<0.01; *** p<0.001).

### EXPERIMENTAL SECTION

1. Morgana is Secreted by Cancer Cells

Morgana is known in the literature as a cytosolic protein expressed in a ubiquitous manner. Studies performed on cancer cells report that Morgana plays an important role as a regulator of several intracellular signaling cascades (2-4).

The inventors highlighted the presence of Morgana in the conditioned medium of various human and murine cancer cells (including breast, lung, colon and melanoma cancer cell lines), but not in non-tumor cell lines, such as the MCF-10A mammary epithelium line (FIG. 1a). Furthermore, other cytoplasmic proteins, even abundant ones, are not detectable in the extracellular medium, which excludes the possibility that the cytoplasmic content is released following cell damage (FIG. 1b). By analyzing the amino acid sequence of Morgana, the inventors excluded the presence of a localization sequence in the endoplasmic reticulum. Furthermore, Morgana secretion is not blocked by treatment with Brefeldin A, an inhibitor of the canonical secretory pathway mediated by the endoplasmic reticulum and Golgi apparatus (FIG. 1c). These results indicate that Morgana is secreted via an unconventional route. It is known that tumor cells are subjected to a large number of factors that induce cellular stress, which activates a particular unconventional protein secretion program (5, 6). In this type of protein secretion, specific proteins lacking a signal peptide can translocate across the plasma membrane using different mechanisms such as release through exosomes, formation of pores across the membrane or secretion via vesicles not deriving from the Golgi apparatus. The molecular mechanisms underlying these events are still largely unknown (5, 6). The extracellular Morgana (eMorgana), at least in part, is not included in vesicular structures and is present free in the culture medium, as it can be immunoprecipitated from tumor cell culture media without using detergent (FIG. 1d). In support of the experimental data obtained by the inventors, the analysis of a database of tumor cell secretomes (7) indicates that Morgana is secreted by relevant numbers of tumor cells of different types, including breast cancer cells (FIG. 1e).

Morgana is known to bind to the HSP90 chaperone protein in the cytoplasm, acting as its co-chaperone (8). It has previously been shown that HSP90 is secreted by tumor cells and that, from the extracellular compartment, it carries out a pro-tumor action, inducing migration and invasion (9). Co-immunoprecipitation analysis performed on the conditioned medium of MDA-MB-231 tumor cells, identified that Morgana and HSP90 interact in the extracellular compartment (FIG. 1f).

Materials and Methods

Conditional Medium Collection

For the collection of the conditioned medium 2×10⁶ cells are plated. After 24 hours the medium is changed to 10 ml of serum-free medium which is collected after 48 hours. The medium is then concentrated with the use of Vivaspin® 20, 10 kDa MWCO up to 1 ml, of which 40 μl are analyzed by Western blotting.

Treatment with BFA

For Brefeldin A treatment, the protocol for the conditioned medium collection was used. In this case, however, the cells were deprived of serum in the presence or absence of Brefeldin A (10 μg/ml) for 5 hours. The medium was then concentrated and analyzed by Western blotting.

Immunoprecipitation from Extract and Conditioned Medium

The cells under study were cold lysed in a lysis buffer containing 1% Triton, Complete 25× protease inhibitors (Roche Applied Science, Indianapolis, IN) and 1 mM phenylmethylsulfonylfluoride and phosphatase inhibitors (10 mM sodium fluoride and 1 mM sodium orthovanadate). After 15 minutes of incubation on the rocker at 4° C., the lysates were centrifuged at 13000 rpm for 15 minutes at 4° C.

The aforementioned collection protocol was followed to immunoprecipitate Morgana from the conditioned medium. Once 1 ml of concentrated medium was obtained, the antibody capable of immunoprecipitating Morgana or control IgG was added. The conditioned medium or the protein lysate were incubated in the presence of the antibodies for one night and the following day resin linked to G protein was added for one hour. The resin was washed 3 times with Tris-buffered saline alone (TBS, 50 mM Tris-Cl, pH 7.5, 150 mM NaCl) respectively in the case of the conditioned medium and 5 times with the lysis buffer in the case of the cell lysate. Immunoprecipitations were then analyzed by Western blotting.

Cell Cultures

Human breast cancer cells MDA-MB-231 (ATCC® number: HTB-26) and BT-549 (ATCC® number: HTB-122) and lung cancer Calu-1 (ATCC® number: HTB-54) and mouse colon cancer cells CT26 (ATCC® number: CRL-2638) were purchased from ATCC. E0771 mouse breast cancer cells were purchased from Tebu-bio (catalog number: 940001-A). MDA-MB-231 were cultured in Dulbecco's Modified Eagle Medium (DMEM, Gibco, Carlsbad, CA) supplemented with 10% fetal bovine serum (FBS, Gibco, Carlsbad, CA) and 5 mM penicillin/streptomycin (Gibco, Carlsbad, CA). CT26 and Calu-1 were cultured in RPMI 1640 (Gibco, Carlsbad, CA) supplemented with 10% fetal bovine serum (FBS, Gibco, Carlsbad, CA) and 5 mM penicillin/streptomycin (Gibco, Carlsbad, CA). BT-549 cells were maintained in RPMI 1640 (Gibco, Carlsbad, CA) supplemented with 0.1% insulin (Sigma Aldrich), 10% fetal bovine serum (FBS, Gibco, Carlsbad, CA) and 5 mM penicillin/streptomycin (Gibco, Carlsbad, CA). E0771 cells were maintained in RPMI 1640 (Gibco, Carlsbad, CA) supplemented with 10 mM Hepes (Gibco, Carlsbad, CA), 10% fetal bovine serum (FBS, Gibco, Carlsbad, CA) and 5 mM penicillin/streptomycin (Gibco, Carlsbad, CA).

Protein Extraction

The cells under study were cold lysed in a lysis buffer containing 1% Triton, Complete 25×protease inhibitors (Roche Applied Science, Indianapolis, IN) and 1 mM phenylmethylsulfonylfluoride and phosphatase inhibitors (10 mM sodium fluoride and 1 mM sodium orthovanadate). After 15 minutes of incubation on the rocker at 4° C., the lysates were centrifuged at 13000 rpm for 15 minutes at 4° C. and were used for Western blot analysis.

2. Extracellular Morgana Induces Tumor Cell Migration

Infection of human breast cancer cells MDA-MB-231 and BT-549 with two different shRNAs against Morgana induces 85% and 80% protein depletion, respectively (3). Morgana-depleted cells did not show growth differences in vitro (2, 3) when compared with control cells (infected with an empty vector). Morgana-depleted MDA-MB-231 and BT-549 cells migrate significantly less than controls when subjected to a wound healing migration test (FIG. 2a-b). To analyze the role of eMorgana in tumor cell migration, the inventors produced two recombinant proteins: the maltose binding protein (MBP) and the MBP fused to the Morgana amino acid sequence (MBP-Morgana) in a strain of E. coli (ClearColi® BL21) which produces a mutated form of the lipopolysaccharide (LPS) unable to activate an endotoxic response in mammalian cells. These proteins were added to the culture medium of the Morgana-depleted MDA-MB-231 and BT-549 cells during a wound healing migration test. The result shows that treatment with MBP-Morgana, but not with MBP, is able to recover the migration defect of tumor cells due to the lack of production of the Morgana protein (FIG. 2a-b). The pro-migratory effect of recombinant Morgana is also confirmed in human lung cancer cells Calu-1 (FIG. 2c). Taken together, these results indicate that eMorgana induces cancer cell migration.

Materials and Methods

Wound Healing Migration Assay

The wound healing migration assay is performed by plating 1,500,000 MDA-MB-231 and CALU-1 or 900,000 BT-549 cells in 6-well multiwells. The next day a wound is made inside the well and the cells are kept in culture with serum-free medium treated or not with MBP-Morgana or MBP recombinant proteins. Using the Zeiss inverted microscope and AxioVision software, pictures are taken at time zero and after 24 hours and wound measurements are made to assess the ability of the cells to migrate.

3. Extracellular Morgana Induces Cancer Cell Migration Via Toll-Like Receptors 2 and 4 and LRP1

Immunofluorescence analysis with an anti-MBP antibody on cells interfered for Morgana and treated with the MBP or MBP-Morgana recombinant proteins highlighted the ability of MBP-Morgana, and not of MBP, to bind to defined areas of the cell membrane (FIG. 3a). This result suggests that Morgana carries out its pro-migratory activity by binding membrane receptors. HSP90 is known to be secreted non-canonically by tumor cells and to bind specific receptors on the extracellular side. Toll-like receptors and the LRP1 receptor have been described as receptors for extracellular HSP90 (5).

Migration assays demonstrated that MDA-MB-231 and BT-549 cells interfered for Morgana and treated with MBP-Morgana do not recover their migratory capacity in the presence of antibodies blocking TLR-2 and 4, while an antibody blocking TLR-5 has no effect (FIG. 3b-c). These results demonstrate that Morgana induces migration of tumor cells by binding to TLR-2 and 4 receptors. Furthermore, depletion of LRP1 using shRNA prevents the recovery of migratory activity induced by recombinant Morgana in Morgana-depleted cells (FIG. 3d). These results indicate that TLR-2, TLR4 and LRP1 receptors are necessary for transducing the pro-migratory signal induced by extracellular Morgana within the cells and suggests the existence of a cross-talk between these receptors.

To evaluate the relevance of extracellular Morgana in inducing tumor cell migration in vivo, NOD-scid IL2rgnull (NSG) mice with MDA-MB-231 derived tumors were subjected to intratumoral injections of recombinant Morgana or MBP as a control (100 μg/mouse/injection, every other day). After 4 treatments, mice injected with recombinant Morgana showed twice as many circulating tumor cells in the blood compared to control mice (FIG. 3e). This result indicates that extracellular Morgana promotes the migration of tumor cells also in vivo.

Materials and Methods

Membrane Immunofluorescence

30,000 MDA-MB-231 shMORG cells were plated in 24 multiwells. The cells were treated with MBP or MBP-MORGANA (0.1 μM) and fixed after 24 hours with a 4% paraformaldehyde solution in PBS. The cells were subjected to saturation in 1% BSA in TBS and incubated for 2 hours with a primary antibody capable of recognizing MBP, without using detergents for membrane permeabilization. Cells were incubated for 1 hour with Alexa Fluor® 647 secondary antibody and 4′,6-diamidine-2-phenylindole (DAPI) dye.

Wound Healing Assay with TLR Blocking Antibodies

MDA-MB-231 and BT-549 cells were plated (1.5×10⁶ and 9×10⁵ respectively) in 6 multiwells and after 24 hours a wound was performed. Cells were treated with recombinant MBP or MBP-MORGANA proteins (0.1 μM) alone or in combination with TLR2 (30 ng/ml), TLR4 (100 μM) or TLR5 (100 μM) antibodies. Pictures were taken at time 0 and after 24 hours under a Zeiss microscope (Carl Zeiss). The percentage of wound closure was calculated with Axio Vision.

Circulating Tumor Cell Evaluation Assay

1×10⁶ MDA-MB-231 cells infected with a GFP-containing vector were inoculated into 7-week-old immunodeficient NSG mice. After tumor growth (3 weeks later), the animals were treated with vehicle or 100 μg of recombinant MBP protein or 100 μg of recombinant MBP-MORGANA protein. The animals were treated 4 times, every other day and after the last treatment the mice were sacrificed and blood collected. Blood samples were analyzed by flow cytometry for the presence of GFP positive cells.

4. Production of Monoclonal Antibodies Against Morgana and Selection of an Antibody Capable of Blocking the Function of Extracellular Morgana

The inventors produced monoclonal antibodies against murine Morgana using the GST-Morgana fusion protein to immunize 2 BALB/c mice. Sera from both mice were tested for ELISA and Western blot and the best was selected.

Amino Acid Sequence of the Morgana Protein:

(SEQ ID NO: 2)
MALLCYNRGCGQRFDPEANSDDACTYHPGVPVFHDALKGWSCCKRRTTDF
SDFLSIVGCTKGRHNSEKPPEPVKPEVKTTEKKELSELKPKFQEHIIQAP
KPVEAIKRPSPDEPMTNLELKISASLKQALDKLKLSSGSEEDKKEEDSDE
IKIGTSCKNGGCSKTYQGLQSLEEVCVYHSGVPIFHEGMKYWSCCRRKTS
DFNTFLAQEGCTRGKHVWTKKDAGKKVVPCRHDWHQTGGEVTISVYAKNS
LPELSQVEANSTLLNVHIVFEGEKEFHQNVKLWGVIDVKRSYVTMTATKI
EITMRKAEPMQWASLELPTTKKQEKQKDIAD

The ability of the antibody to recognize the Morgana protein was first analyzed using an ELISA test against the recombinant MBP-Morgana protein. At the end of this preliminary analysis, 10 clones were considered positive and kept in culture for three weeks. An ELISA test was carried out every week to confirm their positivity. At the end of the analysis, the clones that maintained a good expression were tested for Western blot, immunoprecipitation and immunofluorescence. The 6 best clones were then subcloned by limiting dilutions. The medium of the selected subclones was re-analyzed in Western blot and the positive subclones capable of providing the strongest signals were tested to evaluate their ability to block the migration of human triple negative breast cancer cells (MDA-MB-231 and BT-549) and lung cancer (Calu-1) and compared to antibodies to Morgana previously produced in the laboratory (FIG. 4a-c). Among the antibodies used, the only one to show a blocking effect on migratory activity was the monoclonal antibody 5B11B3 (mAb 5B11B3). This antibody inhibits migration in a dose-dependent manner (FIG. 4a-b). The hybridoma cells that produce mAb 5B11B3 were cultured in a bioreactor and 10-15 ml of supernatant of the hybridoma were collected weekly for about 8-10 weeks by monitoring the concentration of the antibody and the ability to recognize Morgana by Western blot. The collected supernatants were combined, the antibody was purified by means of a sepharose column conjugated to protein A and then stored at −20° C.

Materials and Methods

Production of the Monoclonal Antibody

To produce the monoclonal antibodies against Morgana, 2 BALB/c mice were immunized with repeated intraperitoneum injections of a recombinant protein consisting of the fusion of the glutathione S-transferase (GST) protein with the whole murine Morgana protein (GST-Morgana) emulsified in Freund's complete adjuvant. The reactivity of the serum was analyzed by ELISA test against the recombinant protein MBP-Morgana. The animal showing the best reactivity in this assay was sacrificed and the spleen was used for fusion with NS1 murine myeloma cells, as previously described (Antibodies: A Laboratory Manual, CSH Press, 2014). The clones grown in selection medium and positive in ELISA, Western blot and immunoprecipitation were subsequently subcloned. The hybridoma cells derived from the subclone that produces the antibody able to block cell migration (mAb 5B11B3) were cultured in a CELLine™ 1000 (Wheaton) bioreactor, according to the seller's instructions. 10-15 ml of medium were collected weekly for about 8-10 weeks and the concentration of the antibody present in the medium and its ability to recognize Morgana by Western blot was monitored. At the end of the collections, the collected supernatants were combined, the antibody was purified using a sepharose column conjugated to protein A and then stored at −20° C.

5. Characterization of the Antibody

Using the Thermo Fisher kit (Pierce Rapid Isotyping kit mouse) and the Sigma kit (IsoQuick™ Kits for Mouse Monoclonal Isotyping) the isotype of the 5B11B3 antibody was characterized as IgG1/kappa (FIG. 4d). In all the in vitro and in vivo experiments, a control antibody of the same isotype produced in the same way was therefore used, i.e. by culture in a bioreactor and purified from the culture medium by means of a sepharose column conjugated to protein A.

6. Characterization of the Morgana Epitope Recognized by the Monoclonal Antibody 5B11B3

The epitope recognized by the 5B11B3 antibody was identified by Western blot assays using fragments of the Morgana protein fused to the GST protein. In particular, 8 constructs were created in which 8 sequences encoding different Morgana fragments were obtained by PCR and were fused in frame with the sequence coding for the GST protein in pGEX plasmids. The constructs obtained were sequenced and then transformed into BL21 strain of E. coli acteria for the production of recombinant proteins. Total protein extracts of the bacteria containing the different constructs were analyzed by Western blot. The results indicate that the 5B11B3 antibody recognizes an epitope included in the sequence from amino acid 85 to 110. (FIG. 4e).

The epitope sequence recognized by the 5B11B3 antibody is underlined within the amino acid sequence SEQ ID NO: 2 of the Morgana protein:

MALLCYNRGC GORFDPEANS DDACTYHPGV PVFHDALKGW
SCCKRRTTDF SDFLSIVGCT KGRHNSEKPP EPVKPEVKTT
EKKELSELKP KFQEHIIQAP KPVEAIKRPS PDEPMTNLEL
KISASLKQAL DKLKLSSGSE EDKKEEDSDE IKIGTSCKNG
GCSKTYQGLQ SLEEVCVYHS GVPIFHEGMK YWSCCRRKTS
DENTFLAQEG CTRGKHVWTK KDAGKKVVPC RHDWHQTGGE
VTISVYAKNS LPELSQVEAN STLLNVHIVF EGEKEFHQNV
KLWGVIDVKR SYVTMTATKI EITMRKAEPM QWASLELPTT
KKQEKQKDIA D

Epitope recognized by the 5B 11B3 antibody: LSELKP KFQEHIIQAP KPVEAIKRPS (SEQ ID NO: 1).

CDR Sequences

RNA was isolated from hybridoma cells following the TRIzol® Reagent technical manual. Total RNA was then retrotranscribed into cDNA using anti-sense primers or isotype-specific universal primers following the FirstScript™ 1st Strand cDNA synthesis kit technical manual. Regions encoding the antibody fragments of VH, VL, CH and CL were amplified according to the GenScript Rapid Extremity Amplification (RACE) standard operating procedure. The sequences encoding the amplified antibody fragments were cloned separately into a standard cloning vector. PCR of single colonies was performed to screen for clones with correctly sized inserts. No less than five colonies with correctly sized inserts were sequenced for each fragment.

The sequences of different clones were aligned to obtain the final sequence.

Heavy Chain: DNA Sequence (1374 bp):

Signal sequence-FR1-CDR1-FR2-CDR2-FR3-CDR3-FR4-
Constant region-Stop codon
(SEQ ID NO: 13)
ATGGGATGGAGCTGTATCATCCTCTTCTTGGTATCAACAGCTACAGGTGT
CCACTCCCAGGTCCAACTGCAGCAGCCTGGGGCTGAGCTTGTGAAGCCTG
GGGCTTCAGTGAAGCTGTCCTGCAAGGCTTCTGGCTACACCTTCACCTCC
TACTGGATGCACTGGGTGAAACAGAGGCCTGGACAAGGCCTTGAGTGGAT
CGGACAGATTGATCCTTCTGATAATTATACTAGCTACAATCAAAAATTCA
AGGGCAAGGCCACATTGACTGTAGACACATCCTCCAGCACAGCCTACATG
CAACTCAGCAGCCTGACATCTGAGGATTCTGCGGTCTATTACTGTGCAAG
TCCGTATGGTAGTTACTGGGGCCAAGGGACTCTGGTCACTGTCTCTGCAT
CCAAAACGACACCCCCATCTGTCTATCCACTGGCCCCTGGATCTGCTGCC
CAAACTAACTCCATGGTGACCCTGGGATGCCTGGTCAAGGGCTATTTCCC
TGAGCCAGTGACAGTGACCTGGAACTCTGGATCCCTGTCCAGCGGTGTGC
ACACCTTCCCAGCTGTCCTGCAGTCTGACCTCTACACTCTGAGCAGCTCA
GTGACTGTCCCCTCCAGCACCTGGCCCAGCGAGACCGTCACCTGCAACGT
TGCCCACCCGGCCAGCAGCACCAAGGTGGACAAGAAAATTGTGCCCAGGG
ATTGTGGTTGTAAGCCTTGCATATGTACAGTCCCAGAAGTATCATCTGTC
TTCATCTTCCCCCCAAAGCCCAAGGATGTGCTCACCATTACTCTGACTCC
TAAGGTCACGTGTGTTGTGGTAGACATCAGCAAGGATGATCCCGAGGTCC
AGTTCAGCTGGTTTGTAGATGATGTGGAGGTGCACACAGCTCAGACGCAA
CCCCGGGAGGAGCAGTTCAACAGCACTTTCCGCTCAGTCAGTGAACTTCC
CATCATGCACCAGGACTGGCTCAATGGCAAGGAGTTCAAATGCAGGGTCA
ACAGTGCAGCTTTCCCTGCCCCCATCGAGAAAACCATCTCCAAAACCAAA
GGCAGACCGAAGGCTCCACAGGTGTACACCATTCCACCTCCCAAGGAGCA
GATGGCCAAGGATAAAGTCAGTCTGACCTGCATGATAACAGACTTCTTCC
CTGAAGACATTACTGTGGAGTGGCAGTGGAATGGGCAGCCAGCGGAGAAC
TACAAGAACACTCAGCCCATCATGGACACAGATGGCTCTTACTTCGTCTA
CAGCAAGCTCAATGTGCAGAAGAGCAACTGGGAGGCAGGAAATACTTTCA
CCTGCTCTGTGTTACATGAGGGCCTGCACAACCACCATACTGAGAAGAGC
CTCTCCCACTCTCCTGGTAAATGA

Heavy Chain: Amino Acidic Sequence (457 Aa)

Signal peptide-FR1-CDR1-FR2-CDR2-FR3-CDR3-FR4-
Constant region-Stop codon
(SEQ ID NO: 14)
MGWSCIILFLVSTATGVHSQVQLQQPGAELVKPGASVKLSCKASGYTFTS
YWMHWVKQRPGQGLEWIGQIDPSDNYTSYNQKFKGKATLTVDTSSSTAYM
QLSSLTSEDSAVYYCASPYGSYWGQGTLVTVSASKTTPPSVYPLAPGSAA
QTNSMVTLGCLVKGYFPEPVTVTWNSGSLSSGVHTFPAVLQSDLYTLSSS
VTVPSSTWPSETVTCNVAHPASSTKVDKKIVPRDCGCKPCICTVPEVSSV
FIFPPKPKDVLTITLTPKVTCVVVDISKDDPEVQFSWFVDDVEVHTAQTQ
PREEQFNSTFRSVSELPIMHQDWLNGKEFKCRVNSAAFPAPIEKTISKTK
GRPKAPQVYTIPPPKEQMAKDKVSLTCMITDFFPEDITVEWQWNGQPAEN
YKNTQPIMDTDGSYFVYSKLNVQKSNWEAGNTFTCSVLHEGLHNHHTEKS
LSHSPGK-
(SEQ ID NO: 3)
CDRH1: SYWMH
(SEQ ID NO: 4)
CDRH2: QIDPSDNYTSYNQKFKG
(SEQ ID NO: 5)
CDRH3: PYGSY

Heavy chain variable region (FR1-CDR1-FR2-CDR2-FR3-CDR3-FR4):

(SEQ ID NO: 9)
QVQLQQPGAELVKPGASVKLSCKASGYTFTSYWMHWVKQRPGQGLEWIGQ
IDPSDNYTSYNQKFKGKATLTVDTSSSTAYMQLSSLTSEDSAVYYCASP
YGSYWGQGTLVTVSA

Heavy chain (FR1-CDR1-FR2-CDR2-FR3-CDR3-FR4-Costant region):

(SEQ ID NO: 10)
QVQLQQPGAELVKPGASVKLSCKASGYTFTSYWMHWVKQRPGQGLEWIGQ
IDPSDNYTSYNQKFKGKATLTVDTSSSTAYMQLSSLTSEDSAVYYCASPY
GSYWGQGTLVTVSASKTTPPSVYPLAPGSAAQTNSMVTLGCLVKGYFPEP
VTVTWNSGSLSSGVHTFPAVLQSDLYTLSSSVTVPSSTWPSETVTCNVAH
PASSTKVDKKIVPRDCGCKPCICTVPEVSSVFIFPPKPKDVLTITLTPKV
TCVVVDISKDDPEVQFSWFVDDVEVHTAQTQPREEQFNSTFRSVSELPIM
HQDWLNGKEFKCRVNSAAFPAPIEKTISKTKGRPKAPQVYTIPPPKEQMA
KDKVSLTCMITDFFPEDITVEWQWNGQPAENYKNTQPIMDTDGSYFVYSK
LNVQKSNWEAGNTFTCSVLHEGLHNHHTEKSLSHSPGK

Light chain: DNA sequence (717 bp)

Signal sequence-FR1-CDR1-FR2-CDR2-FR3-CDR3-FR4-
Constant region-Stop codon
(SEQ ID NO: 15)
ATGAAGTTGCCTGTTAGGCTGTTGGTGCTGATGTTCTGGATTCCTGCTTC
CAGCAGTGATGTTGTGATGACCCAAACTCCACTCTCCCTGCCTGTCAGTC
TTGGTGATCAAGCCTCCATCTCTTGCAGATCTAGTCAGAGCCTTGTACAC
AGTAATGGAAACTCCTATTTACATTGGTACCTGCAGAACCCAGGCCAGTC
TCCAAAGCTCCTGATCTACAAAGTTTCCAACCGATTTTCTGGGGTCCCAG
ACAGGTTCAGTGGCAGTGGATCAGGGACAGATTTCACACTCAAGATCAGC
AGAGTGGAGGCTGAGGATCTGGGAGTTTATTTCTGCTCTCAAAGTACACA
TGTTCCGCTCACGTTCGGTGCTGGGACCAAGCTGGAGCTGAAACGGGCTG
ATGCTGCACCAACTGTATCCATCTTCCCACCATCCAGTGAGCAGTTAACA
TCTGGAGGTGCCTCAGTCGTGTGCTTCTTGAACAACTTCTACCCCAAAGA
CATCAATGTCAAGTGGAAGATTGATGGCAGTGAACGACAAAATGGCGTCC
TGAACAGTTGGACTGATCAGGACAGCAAAGACAGCACCTACAGCATGAGC
AGCACCCTCACGTTGACCAAGGACGAGTATGAACGACATAACAGCTATAC
CTGTGAGGCCACTCACAAGACATCAACTTCACCCATTGTCAAGAGCTTCA
ACAGGAATGAGTGTTAG

Light Chain: Amino Acid Sequence (238 Aa)

Signal peptide-FR1-CDR1-FR2-CDR2-FR3-CDR3-FR4-
Constant region-Stop codon
(SEQ ID NO: 16)
MKLPVRLLVLMFWIPASSSDVVMTQTPLSLPVSLGDQASISCRSSQSLVH
SNGNSYLHWYLQNPGQSPKLLIYKVSNRFSGVPDRFSGSGSGTDFTLKIS
RVEAEDLGVYFCSQSTHVPLTFGAGTKLELKRADAAPTVSIFPPSSEQLT
SGGASVVCFLNNFYPKDINVKWKIDGSERQNGVLNSWTDQDSKDSTYSMS
STLTLTKDEYERHNSYTCEATHKTSTSPIVKSFNRNEC-
(SEQ ID NO: 6)
CDRL1: RSSQSLVHSNGNSYLH
(SEQ ID NO: 7)
CDRL2: KVSNRFS
(SEQ ID NO: 8)
CDRL3: SQSTHVPLT
Light chain variable region
(FR1-CDR1-FR2-CDR2-FR3-CDR3-FR4):
(SEQ ID NO: 11)
DVVMTQTPLSLPVSLGDQASISCRSSQSLVHSNGNSYLHWYLQNPGQSPK
LLIYKVSNRFSGVPDRFSGSGSGTDFTLKISRVEAEDLGVYFCSQSTHVP
LTFGAGTKLELKRA
Light chain
(FR1-CDR1-FR2-CDR2-FR3-CDR3-FR4-Constant region):
(SEQ ID NO: 12)
DVVMTQTPLSLPVSLGDQASISCRSSQSLVHSNGNSYLHWYLQNPGQSPK
LLIYKVSNRFSGVPDRFSGSGSGTDFTLKISRVEAEDLGVYFCSQSTHVP
LTFGAGTKLELKRADAAPTVSIFPPSSEQLTSGGASVVCFLNNFYPKDIN
VKWKIDGSERQNGVLNSWTDQDSKDSTYSMSSTLTLTKDEYERHNSYTCE
ATHKTSTSPIVKSFNRNEC

7. Safety Tests of the 5B11B3 Antibody on Healthy Mice

Mice of the C57/BL6 strain were injected intravenously with 100 μg of monoclonal antibody 5B11B3 or a control antibody three times a week for a month to test antibody toxicity. At the end of the treatment, the mice were sacrificed and blood and organs were collected for analysis. The tests carried out on the blood of mice are summarized in the table below. The values obtained in the mice treated with the 5B 11B3 antibody do not appear to be significantly different from the values of the control group and show that the treatment does not alter pancreatic, hepatic and renal function. Histopathological analyzes did not reveal the presence of morphological alterations or inflammatory infiltrates in all the organs examined (liver, kidneys, lungs).

| | Amylase | AST | ALT | Creatinin | UREA |
|---|---|---|---|---|---|
| | (UI) | (UI) | (UI) | (mg/dl) | (mg/dl) |
| CONTROL | 417 ± 23 | 175 ± 11 | 38 ± 3 | 0.28 ± 0.1 | 68 ± 9 |
| IgG | | | | | |
| 5B11B3 | 361 ± 18 | 160 ± 27 | 46 ± 7 | 0.36 ± 0.06 | 46 ± 3 |

Materials and Methods

Animals

Wild type C57BL/6 animals were treated for one month with intravenous injections of 100 μg of 5B11B3 monoclonal antibody or control IgG. At the end of the experiment, the animals were sacrificed, the plasma was taken to carry out tests on the levels of metabolites indicating organ damage.

The animals were used according to the guidelines and institutional regulations on animal welfare, approved by the Bioethical Committee of the Ministry of Health (authorization n.540/2018-PR issued on 16 Jul. 2018).

8. The 5B11B3 Monoclonal Antibody is Able to Inhibit the Growth of Breast Tumors and the Formation of Metastases in Mouse Models

As a first and quick attempt to test the ability of mAb 5B11B3 to inhibit tumor cell migration, the inventors exploited an experimental model of metastasis in immunocompromised mice. The formation of metastases in this test depends primarily on the ability of cancer cells to survive in the bloodstream, to migrate across the endothelial barrier and to proliferate in the secondary organ. It should be noted that mAb 5B11B3 does not alter the viability and proliferation of tumor cells in vitro (FIG. 5a-b). NSG mice were injected intravenously with 500,000 human breast cancer cells MDA-MB-231 and, starting the next day, treated with mAb 5B11B3 or control antibody twice per week (100 μs via intravenous injection).

Lung metastatic burden after 2 weeks from tumor cell injection was significantly lower in mice treated with mAb 5B11B3 compared to controls (FIG. 5c).

The efficacy of mAb 5B11B3 was then tested in immunocompetent mouse models generated by subcutaneous injection of E0771 mouse breast cancer cells, which secrete a relevant amount of Morgana (FIG. 1a). Starting the day after the injection of the cells, the mice were treated with mAb 5B11B3 or IgG as a control (100 μg, IV injection, 3 times a week). After 20 days, the animals treated with mAb 5B11B3 showed a consistent reduction in the growth of the primary tumor (FIG. 5d).

To investigate the presence of possible off-target activities of mAb 5B11B3 responsible for reduced tumor growth, the inventors generated synergistic mouse models using 4T1 breast cancer cells, expressing high Morgana levels but secreting a very low amount of the protein (FIG. 1a). In this model, treatment with mAb 5B11B3 (100 μg) did not reduce tumor volume compared to treatment with control IgG (FIG. 5e), supporting the idea that Morgana is the therapeutic target of mAb 5B11B3. To assess the therapeutic potential of mAb 5B11B3 treatment, the inventors set up a curative protocol in which treatments with mAb 5B11B3 or control IgG (100 μg by intravenous injection, three times a week) began when the tumors became palpable (15 mm³) and lasted for 4 weeks. Treatment with mAb 5B11B3, but not with the control antibody, caused a significant inhibition of primary tumor growth (FIG. 5f) and a drastic reduction in lung metastases (FIG. 5g).

Materials and Methods

Proliferation Assays

Cells (MDA-MB-231) were plated (10,000) in 96 multiwells. They were treated with mAb 5B11B3, control IgG antibody or PBS in the medium in the presence of serum. The treatments were stopped at different times (24, 48, 72, 96, 120, 144 hours), fixing the cells with 4% paraformaldehyde in PBS. Cells were stained with Crystal violet. For the quantification of the cells, the dye retained by the cells was solubilized in a 60% Acetic Acid solution and absorbance was read at 600 nm.

Apoptosis Assays

Cells (MDA-MB-231) were plated in 12-well multiwells. They were treated with mAb 5B11B3, control IgG antibody or PBS in the medium in the presence of serum. The treatments were stopped at different times (24, 48 h) and the percentage of apoptotic cells was evaluated by labeling with Annexin V and propidium iodide and by flow cytometric analysis.

Treatment of the Mouse Model of Metastatic Breast Cancer with the 5B11B3 Antibody.

NSG mice were injected intravenously with 500,000 MDA-MB-231 human breast cancer cells and, starting the next day, treated with mAb 5B11B3 or control antibody twice a week (100 μg per injection intravenous) for two weeks.

Female wild type C57BL/6 mice were subcutaneously inoculated with 200,000 E0771 tumor cells. Two days after inoculation, treatment with 100 μg of 5B11B3 monoclonal antibody or control IgG was started intravenously three times a week. Female wild type C57BL/6 and BALB/c mice were subcutaneously inoculated with 200,000 E0771 and 4T1 tumor cells, respectively. In a first experimental approach, the treatment of the animals with the antibodies began the day after cancer cell inoculation. The treatment was carried out with 100 μg of monoclonal antibody 5B11B3 or control IgG (of the same isotype IgG1/kappa) intravenously three times a week. The animals were sacrificed at 20 and 15 days after inoculation of the tumor cells, respectively (after 8 and 7 treatments) and the tumor weight was assessed. In a second approach, treatments with the antibodies began only when the tumor derived from E0771 cells was palpable and with volume equal to 15 mm³. The treatment was carried out with 100 μg of monoclonal antibody 5B11B3 or control IgG (of the same isotype IgG1/kappa) intravenously three times a week. During the experiment tumor growth was measured using a caliper. The animals were sacrificed at 42 days (after 10 treatments), the tumor was measured and lungs were collected to assess the number of metastases. The lungs were left overnight in 4% paraformaldehyde in PBS and then transferred to 75% ethanol. Lungs were then paraffin-embedded, microtome cut and the sections stained with hematoxylin and eosin.

The animals were used according to the guidelines and institutional regulations on animal welfare, approved by the Bioethical Committee of the Ministry of Health (authorization n.540/2018-PR issued on 16 Jul. 2018).

9. Treatment with the 5B11B3 Antibody Causes a Greater Recruitment of Macrophages and CD8 Positive T Lymphocytes in the Primary Tumor

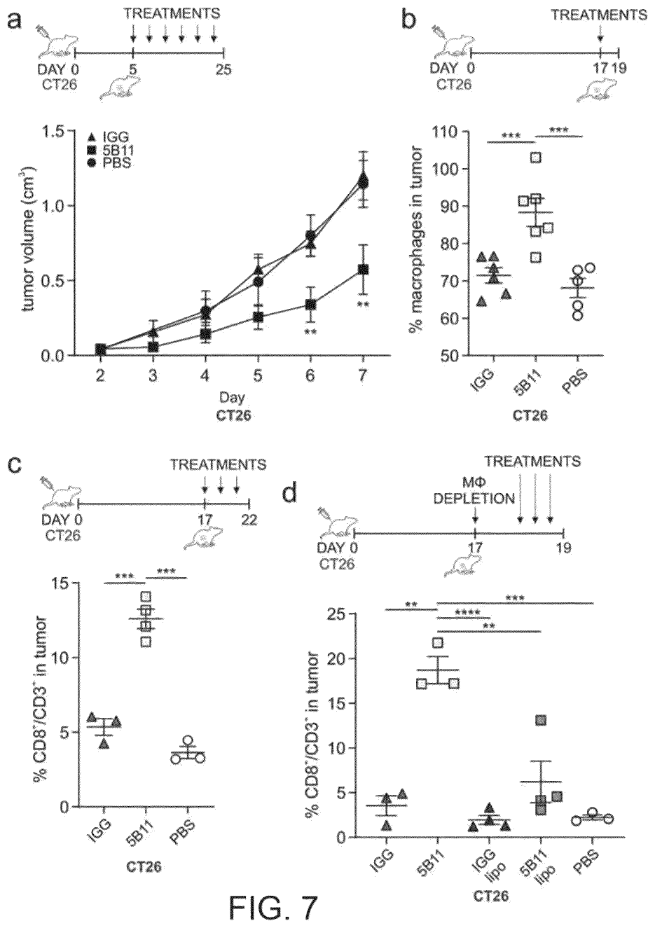

To study the mechanism of action of the monoclonal antibody 5B11B3, in vitro assays were initially performed demonstrating that treatment with mAb 5B11B3 does not cause differences in the proliferation (FIG. 6a) and apoptosis (FIG. 6b) of E0771 breast cancer cells. Furthermore, treatment of immunocompromised mice carrying MDA-MB-231-derived tumors with the 5B11B3 antibody has no effect on tumor growth, suggesting an involvement of the immune system in the ability of mAb 5B11B3 to reduce tumor growth in vivo (FIG. 6c). Some monoclonal antibodies are able to cause a reduction in tumor progression by activating the immune system response through the mechanisms of ADCC (Antibody-Dependent Cell mediated Cytotoxicity, in which the immune response mediators are Natural Killer cells), CDC (Complement-Dependent Cytotoxicity, in which the mediator of the immune response is the complement system) and ADPh (Antibody-Dependent cellular Phagocytosis, in which the mediators of the immune response are macrophages). In vitro co-culture experiments excluded the role of NK cells (FIG. 6d), while they demonstrated a greater ability of primary macrophages to engulf E0771 tumor cells in the presence of mAb 5B11B3 compared to control IgG, demonstrating that the antibody acts triggering the phagocytosis of tumor cells by macrophages (ADPh) (FIG. 6e). Analysis of tumor immune composition after a single injection in mice of mAb 5B11B3 (100 μg) showed a significant increase in macrophages compared to mice treated with control IgG (FIG. 60, further suggesting that the 5B11B3 antibody blocks tumor progression via the recruitment of macrophages in the primary tumor. Macrophages are known to produce cytokines capable of recruiting other populations of the immune system that may play a role in tumor shrinkage. To evaluate this possibility, tumors treated three times (every other day) with mAb 5B11B3 or with control IgG were analyzed. The flow cytometer analysis showed a significant increase in CD8+ T lymphocytes in tumors treated with the 5B11B3 antibody (FIG. 6g). To confirm the importance of macrophages in the recruitment of CD8⁺ T lymphocytes, in vivo experiments were performed in which the macrophage population was depleted using clodronate liposomes. The clodronate liposomes were inoculated intraperitoneum into mice with tumor size of 15 mm³, after three days the animals were subjected to three treatments (every other day) with the 5B11B3 or control antibody. At the end of the experiment, the recruitment of CD8⁺ T lymphocytes into the tumor was evaluated (FIG. 6h). As shown in FIG. 6h, in mice in which macrophages have been depleted by clodronate liposomes, CD8+ T lymphocytes do not accumulate in the tumor, indicating that macrophages recruited into the tumor by the 5B11B3 antibody are responsible for subsequent CD8⁺ T lymphocyte recruitment.

Materials and Methods

Proliferation Assays

10,000 E0771 cells were plated in 96 multiwells and were treated with mAb 5B11B3, with the control IgG antibody or PBS in medium in the presence of serum. The treatments were stopped at different times (24, 48, 72, 96, 120, 144 hours), fixing the cells with 4% paraformaldehyde in PBS. Cells were stained with Crystal violet. For the quantification of the cells, the dye retained by the cells was solubilized in a 60% acetic acid solution and absorbance was read at 600 nm.

Apoptosis Assays

E0771 cells were plated in 12-well multiwells. They were treated with mAb 5B11B3, control antibody or PBS in medium in the presence of serum. The treatments were stopped at 24 and 48 h and the percentage of apoptotic cells was evaluated by flow cytometry by labeling with Annexin V and propidium iodide.

Tumor Growth Assays in Immunocompromised Mice

NSG mice were inoculated subcutaneously with 1×10⁶ MDA-MB-231. Starting from the twentieth day after inoculation, the animals were treated with the monoclonal antibody 5B11B3 or the control antibody (100 μg intravenously three times a week) or PBS. Tumor growth was monitored throughout the course of the experiment with the use of the caliper. At the end of the experiment, the tumor weight was measured.

Antibody Dependent Cell Mediated Cytotoxicity Assay (ADCC)

The ADCC assay was performed by co-culturing E0771 cells with splenocytes obtained from C57BL/6 mice. 1×10⁶ E0771 cells were labeled by incubation in a 2 mM solution of carboxyfluorescein succinimide ester (CFSE) in PBS. Tumor cells and splenocytes were co-cultured in a ratio of 200:1-100:1-50:1. The co-culture was carried out in the presence of the 5B11B3 antibody or collagenase A antibody 1 μg/μ1 for 15 minutes. The red blood cells were then lysed with buffer containing NH₄Cl, KHCO₃, EDTA and water for 5 minutes. The obtained samples were saturated with CD16/32 FC Blocking (Biolegend) for 30 minutes and labeled for 15-30 minutes with the following antibody panels:

| FITC | PE | PERCP | PECY7 | BV421 | BV510 | APC | APCCY7 |
|---|---|---|---|---|---|---|---|
| CD3 | CD49B | 7AAD | B220 | CD8 | CD45 | GAMMA/DELTA | CD4 |
| CD11B | CD206 | 7AAD | F4/80 | LY6G | CD45 | MHCII | LY6C |

The two flow cytometric analyses allowed to evaluate the presence of the following immune cells:

Panel 1: CD8⁺ T lymphocytes, CD4⁺ T lymphocytes, Natural Killer cells, Gamma Delta T lymphocytes and B lymphocytes.

Panel 2: M1, M2 macrophages, neutrophils and Myeloid Derived Suppressor Cells

7AAD was used to limit the analysis to live CD45⁺ cell population.

Analyzes were performed with a BD FACSVerse flow cytometer.

Macrophage depletion was performed using clodronate liposomes (Liposome BV, Amsterdam, The Netherlands). Liposomes were inoculated intraperitoneum 100 μg of suspension/10 g of animal weight. Depletion was done in mice with tumors of 15 mm³ size. The effectiveness of the treatment with clodronate liposomes was verified 3 days after the injection of the liposomes by flow cytometric analysis. Three days after the administration of the liposomes, the mice were subjected to 3 treatments with mAb 5B11B3 or with the control antibody. Lymphocyte recruitment in tumors was evaluated by flow cytometric analysis at the end of treatment.

The animals were used according to the guidelines and institutional regulations on animal welfare, approved by the Bioethical Committee of the Ministry of Health (authorization n.540/2018-PR issued on 16 Jul. 2018).

10. The 5B11B3 Monoclonal Antibody is Able to Inhibit the Growth of Colon Tumors in Mouse Models

To evaluate the efficacy of the monoclonal antibody 5B11B3 in blocking the progression of colon cancer, mouse colon cancer cells CT26 were used. CT26 cells secrete Morgana in the conditioned medium (FIG. 1a). BALB/c mice were inoculated subcutaneously with CT26 cells and when the tumor reached the size of 15 mm³ treatments with the monoclonal antibody 5B11B3 and control IgG were started. Six treatments were performed every other day and the animals were sacrificed twenty days after the first treatment. At the end of the experiment, a significant reduction in tumor volume was observed in mice treated with the monoclonal antibody 5B11B3 compared to the control IgG (FIG. 7a). Furthermore, also in this model, the analysis of the tumor immune infiltrate after one and three treatments (every other day) with 5B11B3 revealed a significant increase in macrophages and CD8⁺ T lymphocytes, respectively (FIG. 7b-c). Again, if the macrophage population was depleted in vivo using clodronate liposomes, CD8⁺ T lymphocytes did not accumulate in the tumor following antibody 5B11B3 treatment (FIG. 7d). Overall, these data indicate that the anti-Morgana 5B11B3 monoclonal antibody is able to block tumor growth by recruiting macrophages, which, in turn, recruit CD8⁺ T lymphocytes and that this mechanism of action is effective in preclinical models of different types of tumors.

Materials and Methods

Animals

Female wild type BALB/c mice were subcutaneously inoculated with 200,000 CT26 tumor cells. Five days after inoculation, the animals were treated with 100 μg of 5B11B3 monoclonal antibody or control antibody. The treatments were carried out intravenously three times a week. To evaluate the effects of the antibody on tumor growth, the animals were sacrificed at 25 days (after six treatments). For the evaluation of tumor infiltrate, the animals were sacrificed after one or three treatments. In both cases, tumors were dissociated using mechanical and enzymatic methods, using a lancet and a treatment with collagenase A, 1 μg/μl (Roche Applied Science, Indianapolis, IN) for 15 minutes. The red blood cells were then lysed with buffer containing NH₄C1, KHCO₃, EDTA and water for 5 minutes. The samples were saturated with CD16/32 FC Blocking (Biolegend) for 30 minutes and labeled for 15-30 minutes with the following flow cytometry antibody panels:

| FITC | PE | PERCP | PECY7 | BV421 | BV510 | APC | APCCY7 |
|---|---|---|---|---|---|---|---|
| CD3 | CD49B | 7AAD | B220 | CD8 | CD45 | GAMMA/DELTA | CD4 |
| CD11B | CD206 | 7AAD | F4/80 | LY6G | CD45 | MHCII | LY6C |

The two flow cytometric analyzes allow to evaluate the presence of the following immune cells:

Panel 1: CD8⁺ T lymphocytes, CD4⁺ T lymphocytes, Natural Killer cells, Gamma Delta T lymphocytes and B lymphocytes.

Panel 2: M1, M2 macrophages, neutrophils and Myeloid Derived Suppressor Cells

7AAD was used to limit the analysis to the live CD45⁺ cell population.

Analyses were performed with a BD FACSVerse flow cytometer.

Macrophage depletion was performed using clodronate liposomes (Liposome BV, Amsterdam, The Netherlands). Liposomes were inoculated intraperitoneum 100 μg of suspension/10 g of animal weight. Depletion was done in mice with tumors of 15 mm³ size. The effectiveness of the treatment with clodronate liposomes was verified 3 days after the injection of the liposomes by flow cytometric analysis. Three days after the administration of the liposomes, the mice were subjected to 3 treatments with mAb 5B11B3 or with the control antibody. Lymphocyte recruitment in tumors was evaluated by flow cytometric analysis at the end of treatment.

The animals were used according to the guidelines and institutional regulations on animal welfare, approved by the Bioethical Committee of the Ministry of Health (authorization n.540/2018-PR issued on 16 Jul. 2018).

### REFERENCES

1. Safdari Y, Farajnia S, Asgharzadeh M, Khalili M. Antibody humanization methods—a review and update. Biotechnol Genet Eng Rev 29, 175-186 (2013).
2. Ferretti, R. et al. Morgana/chp-1, a ROCK inhibitor involved in centrosome duplication and tumorigenesis. Dev Cell 18, 486-495 (2010).
3. Fusella, F. et al. Morgana acts as a proto-oncogene through inhibition of a ROCK-PTEN pathway. J Pathol 234:152-163 (2014).
4. Fusella, F. et al. The IKK/NF-kappaB signaling pathway requires Morgana to drive breast cancer metastasis. Nat Commun 8, 1636 (2017).
5. Rabouille, C. Pathways of Unconventional Protein Secretion. Trends Cell Biol 27, 230-240 (2017).
6. Calderwood, S. K., Mambula, S. S., Gray, P. J., Jr. & Theriault, J. R. Extracellular heat shock proteins in cell signaling. FEBS Lett 581, 3689-3694 (2007).
7. Feizi, A., Banaei-Esfahani, A. & Nielsen, J. HCSD: the human cancer secretome database. Database (Oxford) 2015, bav051 (2015).
8. Michowski, W. et al. Morgana/CHP-1 is a novel chaperone able to protect cells from stress. Biochim Biophys Acta 1803, 1043-1049 (2010).
9. Wang, X. et al. The regulatory mechanism of Hsp90alpha secretion and its function in tumor malignancy. Proc Natl Acad Sci USA 106, 21288-21293 (2009).

1. A monoclonal antibody or antibody fragment thereof that binds to an epitope of the extracellular Morgana protein, said epitope consisting of the amino acid sequence SEQ ID NO: 1.

2. The monoclonal antibody or antibody fragment thereof according to claim 1, which is of the IgG1 isotype with a κ chain.

3. The monoclonal antibody or antibody fragment thereof according to claim 1, which is an antibody fragment selected from the group consisting of Fab, Fab′, F(ab′)2, scFv, dsFv, ds-scFv, minibodies, diabodies, and dimers, multimers or bispecific antibody fragments thereof.

4. The monoclonal antibody or antibody fragment thereof according to claim 1, which is a humanized monoclonal antibody or a humanized antibody fragment.

5. The monoclonal antibody or antibody fragment thereof according to claim 1, which comprises the following complementarity-determining regions:
CDRH1 having the amino acid sequence SEQ ID NO:3,
CDRH2 having the amino acid sequence SEQ ID NO:4,
CDRH3 having the amino acid sequence SEQ ID NO:5,
CDRL1 having the amino acid sequence SEQ ID NO:6,
CDRL2 having the amino acid sequence SEQ ID NO:7, and
CDRL3 having the amino acid sequence SEQ ID NO:8.

6. The monoclonal antibody or antibody fragment thereof according to claim 1, which comprises the heavy chain variable region of amino acid sequence SEQ ID NO:9.

7. The monoclonal antibody or antibody fragment thereof according to claim 6, which comprises the light chain variable region of amino acid sequence SEQ ID NO: 11.

8. The monoclonal antibody or antibody fragment thereof according to claim 1, which comprises the heavy chain of amino acid sequence SEQ ID NO:10 and the light chain of amino acid sequence SEQ ID NO:12.

9. An isolated nucleic acid comprising a nucleotide sequence coding for the monoclonal antibody or antibody fragment thereof according to claim 1.

10. An expression vector comprising the nucleotide sequence according to claim 9.

11. A host cell comprising the expression vector according to claim 10.

12. A hybridoma producing a monoclonal antibody or antibody fragment thereof as defined in claim 1.

13. A method for inhibiting growth of tumors secreting the extracellular Morgana protein, said method comprising administering to a subject in need thereof the monoclonal antibody or antibody fragment thereof according to claim 1 or an isolated nucleic acid comprising a nucleotide sequence coding therefor.

14. The method of claim 13, wherein the subject is affected by a tumor secreting the extracellular Morgana protein.

15. The method of claim 13, wherein inhibiting comprises inhibiting tumor growth and/or inhibiting metastases formation.

16. The method of claim 14, wherein the tumor is selected from the group consisting of breast tumor, lung tumor, colon cancer and melanoma.

17. The method of claim 16, wherein the tumor is a triple negative breast tumor or a non-small-cell lung cancer.

18. The method of claim 13, wherein the subject is further administered with one or more further anti-tumor agents.

19. A pharmaceutical composition comprising the monoclonal antibody or antibody fragment thereof as defined in claim 1 or an isolated nucleic acid comprising a nucleotide sequence coding therefor, and a pharmaceutically acceptable excipient, vehicle, buffer and/or stabilizer.

20. (canceled)

21. The method of claim 18, wherein the one or more further anti-tumor agents are selected from immune checkpoint inhibitors.
